(19) United States
(12) Patent Application Publication
(10) Pub. No.: US 2025/0228853 A1
(43) Pub. Date: Jul. 17, 2025

(54) Apoptosis Inhibitors

(71) Applicant: Queen's University at Kingston, Kingston (CA)

(72) Inventors: Danchen Wu, Kingston (CA); Michael Wells, Kingston (CA); Stephen Archer, Kingston (CA); Toni Kristian Rantanen, Burlington (CA)

(21) Appl. No.: 19/024,516

(22) Filed: Jan. 16, 2025

Related U.S. Application Data
(60) Provisional application No. 63/693,267, filed on Sep. 11, 2024.
(60) Provisional application No. 63/621,182, filed on Jan. 16, 2024.

(51) Int. Cl. A61K 31/506; A61K 31/085; A61K 31/404; A61K 31/428; A61K 31/4709; A61P 31/14; C07C 43/225; C07D 209/88; C07D 277/64; C07D 401/06; C07D 405/14

(52) U.S. Cl. CPC A61K 31/506; A61K 31/085; A61K 31/404; A61K 31/428; A61K 31/4709; A61P 31/14; C07C 43/225; C07D 209/88; C07D 277/64; C07D 401/06; C07D 405/14

(57) ABSTRACT

Compounds and pharmaceutical compositions include inhibitors of apoptosis inducing factor (AIF). Pharmaceutical compositions for the treatment and/or mitigation of diseases in which increased rates of apoptosis are part of the disease pathophysiology, particularly, but not exclusively, when levels or activities of AIF are increased.

### RELATED APPLICATIONS

This application claims the benefit of the filing date of U.S. Application No. 63/621,182, filed on Jan. 16, 2024, and U.S. Application No. 63/693,267, filed on Sep. 11, 2024, the contents of which are incorporated herein by reference in their entirety.

### FIELD

The invention relates to inhibitors of apoptosis and uses thereof to treat diseases in which apoptosis is increased.

### BACKGROUND

COVID-19 pneumonia patients suffer from lung injury, due to diffuse alveolar damage and hypoxemia. While most infected people are pauci-symptomatic, ˜5% suffer respiratory or multiorgan failure requiring hospitalization and ˜1.5% die. Many viruses, including coronaviruses like SARS-CoV-2, cause apoptosis by targeting mitochondria in host cells. Viral-mitochondrial interactions can result in mitochondrial membrane permeabilization which depolarizes mitochondrial membrane potential and releases apoptosis inducers, like cytochrome c and apoptosis inducing factor (AIF). There is a need for compounds that disrupt apoptosis.

### SUMMARY

In one aspect, the invention provides a compound selected from 2-[(1-methyl-1,4-dihydroquinazolin-2-yl)methyl)-6-(tetrahydro-2H-pyran-4-yl)-5,6-dihydropyrimidin-4 (3H)-one (1), 2-benzhydryl-4,5,7-trifluorobenzo[d]thiazole (2), N,N-diethyl-3-(2-oxo-1,2-dihydroquinoline-4-carbonyl) quinoline-2-carboxamide (3), 5-cyano-11H-benzo[a]carbazol-6-yl trifluoromethanesulfonate (4), 1-(3-bromopropyl)-4-(3-(cyclohexyloxy)propoxy)benzene (5), or 2-(2-(5-(benzyloxy)-1H-indol-3-yl)ethyl) isoindoline-1,3-dione (6), (see structural formulae in Table 1).

In one aspect, the invention provides a pharmaceutical composition comprising a compound selected from the group consisting of 2-[(1-methyl-1,4-dihydroquinazolin-2-yl)methyl)-6-(tetrahydro-2H-pyran-4-yl)-5,6-dihydropyrimidin-4 (3H)-one (1), 2-benzhydryl-4,5,7-trifluorobenzo[d]thiazole (2), N,N-diethyl-3-(2-oxo-1,2-dihydroquinoline-4-carbonyl) quinoline-2-carboxamide (3), 5-cyano-11H-benzo[a]carbazol-6-yl trifluoromethanesulfonate (4), 1-(3-bromopropyl)-4-(3-(cyclohexyloxy)propoxy)benzene (5), and 2-(2-(5-(benzyloxy)-1H-indol-3-yl)ethyl) isoindoline-1,3-dione (6), or a pharmaceutically acceptable salt thereof, for inhibiting apoptosis in a subject.

In one embodiment, the pharmaceutical composition inhibits apoptosis by inhibiting activity of apoptosis inducing factor (AIF) in the subject. In one embodiment of the pharmaceutical composition, the compound is compound (1), (3), or (6), or a pharmaceutically acceptable salt thereof. In one embodiment, the treatment and/or mitigation of one or more of a viral infection including infections mediated by coronaviruses, including SARS-CoV-2 and HCOV-OC43, and Herpes viruses, stroke, coronary heart disease, myocardial infarction, ischemia-reperfusion injuries, as occurs in cardiac arrest, a neurodegenerative disease, Parkinsonism, Huntington's Chorea, Alzheimer's disease, cardiomyopathies (including ischemic, viral and diabetic cardiomyopathy), fatty liver diseases, non-alcoholic fatty liver diseases, and alcohol-related liver disease. In one embodiment, the pharmaceutical composition is for providing cardioprotection. In one embodiment, the pharmaceutical composition inhibits apoptosis by inhibiting activity of apoptosis inducing factor (AIF) in the subject. In one embodiment, the pharmaceutical composition further comprises a pharmaceutically acceptable vehicle. In one embodiment, the pharmaceutically acceptable vehicle is an excipient. In one embodiment, the compound is present in an amount from 1 mg to 1000 mg. In one embodiment, the compound is present in an amount from 5 mg to 500 mg. In one embodiment, the subject is human.

In one aspect, the invention provides a method for inhibiting apoptosis in a subject comprising administering to the subject an effective amount of the pharmaceutical composition of the above aspect. In one embodiment of the method, the compound is compound (1), (3), or (6), or a pharmaceutically acceptable salt thereof. In one embodiment of the method, the subject is human.

In one aspect, the invention provides a method for inhibiting apoptosis inducing factor (AIF) in a subject comprising administering to the subject an effective amount of the pharmaceutical composition of the above aspect. In one embodiment of the method, the compound is compound (1), (3), or (6), or a pharmaceutically acceptable salt thereof. In one embodiment of the method, the subject is human.

In one aspect, the invention provides use of a compound of the above aspect in the preparation of a medicament for treating and/or mitigating one or more of a viral infection, stroke, coronary heart disease, myocardial infarction, neurodegenerative disease, Parkinsonism, Huntington's Chorea, Alzheimer's disease, cardiomyopathy, fatty liver disease, non-alcoholic fatty liver disease, and alcohol-related liver disease, or for providing cardioprotection. In one embodiment, the viral infection is a virus selected from the group consisting of coronavirus, SARS-CoV-2, HCOV-OC43, and Herpes virus. In one embodiment, the cardiomyopathy is ischemic, viral, or diabetic cardiomyopathy.

### BRIEF DESCRIPTION OF THE DRAWINGS

For a better understanding of the invention and to show more clearly how it may be carried into effect, reference will now be made, by way of example, to the accompanying drawings, wherein:

FIG. 1A shows a bar graph depicting cell count for control (ctrl) versus OC43 (Human coronavirus OC43, HCoV-OC43) alone and in combination with specified compounds.

FIG. 1B shows a bar graph depicting TUNEL (terminal deoxynucleotidyl transferase dUTP nick end labeling) positive cells for control versus OC43 alone and in combination with specified compounds. Note that the AIF inhibitors reduce apoptosis (measured using the TUNEL assay) upon challenge with the human coronavirus HCoVOC43.

FIG. 1C shows a bar graph depicting viral load through Relative N expression, which is a PCR measure of Nucleocapsid mRNA for control versus OC43 alone and in combination with specified compounds. Note that the AIF inhibitor 6 lowers the viral titer of HCoVOC43, addressing a potential concern that inhibiting cell death could raise viral titers.

FIG. 2A shows a photograph of murine lungs obtained from mice that had been infected with murine hepatitis virus-1 (MHV-1) and were treated with vehicle only (control) (top lungs) or compound 6 (bottom lungs). Note the AIF inhibitor reduces lung edema.

FIG. 2B shows micro-CT images of the same lungs as in FIG. 2A (control and treated). Note the AIF inhibitor increases lung perfusion.

FIG. 2C shows a bar graph of hemodynamics of right ventricular systolic pressure (RVSP), where the left three bars are from one mouse that was treated with MHV-1 and vehicle only (control); the right three bars are from one mouse that was treated with MHV-1 plus AIF Inhibitor 6.

### DESCRIPTION

The invention provides methods and compounds 1 to 6 (see Table 1) for inhibiting apoptosis and specifically inhibiting apoptosis inducing factor (AIF) in individuals in need thereof. The invention relates to inhibitors of apoptosis and uses thereof to treat diseases in which apoptosis is increased, which includes viral infections such as COVID-19, which is caused by SARS-CoV-2 and other coronaviruses, and ischemic diseases such as myocardial infarction, myocarditis, and ischemia reperfusion injury as occurs with cardiac arrest, acute renal injury and cardiopulmonary bypass. By preserving mitochondrial health these new compounds, which inhibit the apoptosis mediator, apoptosis inducing factor (AIF), also preserve other mitochondrial functions like oxygen sensing.

Methods for inhibiting apoptosis and inhibiting apoptosis inducing factor (AIF) include administering to a patient or animal an effective amount of the AIF inhibitors described herein. Initially, potential AIF inhibitors were identified using in silico screening. That is, virtual screening was performed to identify compounds having a high predicted binding affinity for apoptosis inducing factor (AIF) (see Table 2). Compounds 1 to 6, which had high predicted affinity, then underwent in vitro screening as described herein to confirm their biological activity. Compounds 1 to 6 are as follows: 2-[(1-methyl-1,4-dihydroquinazolin-2-yl)methyl)-6-(tetrahydro-2H-pyran-4-yl)-5,6-dihydropyrimidin-4 (3H)-one (1); 2-benzhydryl-4,5,7-trifluorobenzo[d]thiazole (2); N,N-diethyl-3-(2-oxo-1,2-dihydroquinoline-4-carbonyl) quinoline-2-carboxamide (3); 5-cyano-11H-benzo[a]carbazol-6-yl trifluoromethanesulfonate (4); 1-(3-bromopropyl)-4-(3-(cyclohexyloxy)propoxy)benzene (5); and 2-(2-(5-(benzyloxy)-1H-indol-3-yl)ethyl) isoindoline-1,3-dione (6). Compounds 1, 3, and 6 were particularly effective in the in vitro screening as described herein.

In one aspect, the invention provides a pharmaceutical composition comprising one or more of compounds 1-6, or a pharmaceutically acceptable salt thereof, and a pharmaceutically acceptable vehicle. The pharmaceutical composition is for the treatment and/or mitigation of conditions that involve increased rates of apoptosis, or conditions that involve an increase in apoptosis inducing factor (AIF).

A method of apoptosis inhibition is also provided that includes administering to a patient or animal an effective amount of one or more of compounds 1 to 6. A method of inhibiting apoptosis inducing factor (AIF) is also provided that includes administering to a patient or animal an effective amount of one or more of compounds 1 to 6.

Non-limiting examples of diseases that involve increased rates of apoptosis or an increase in apoptosis inducing factor (AIF), include: stroke, coronary heart disease, neurodegenerative diseases, Parkinsonism, Huntington's Chorea, Alzheimer's disease, diabetic cardiomyopathy, fatty liver diseases, non-alcoholic fatty liver diseases, alcohol-related liver disease, hypoxic-ischemia, stroke, ischemic stroke, coronary heart disease, myocardial infarction, neurodegenerative disorder (e.g., Parkinson's disease, Alzheimer's disease, Huntington's disease, amyotrophic lateral sclerosis), cardiometabolic disease, sepsis, kidney ischemia-reperfusion injury, cardiometabolic disease (e.g., diabetes, obesity, metabolic syndrome, tumor metabolism), cardiomyopathy (e.g., ischemic, viral, diabetic cardiomyopathy), viral infection (e.g., coronavirus, COVID-19, SARS-CoV-2, HCOV-OC43, Reovirus, West Nile Virus, Herpes), or to provide cardioprotection around cardiopulmonary bypass or cardiac arrest.

The term “effective amount” encompasses the term “dose” or “dosage,” and is intended to refer to the quantity of pharmaceutically active ingredient administered to the individual in need thereof capable of producing the desired therapeutic effect. The term may refer to a single one-time dose, in a physically discrete unit, such as, for example, in a pill or injection or may refer to multiple doses in physically discrete units. The term “effective amount” also encompasses the quantity of pharmaceutically active ingredient administered to the individual, expressed as the number of molecules, moles, grams, or volume per unit body mass of the individual, such as, for example, mol/kg, mg/kg, ng/kg, ml/kg, or the like, sometimes referred to as concentration administered. The effective amount of pharmaceutically active ingredient may vary among individuals and may fluctuate within an individual over time, depending on factors such as, but not limited to, the condition being treated, genetic profile, metabolic rate, biotransformation capacity, frequency of administration, formulation administered, elimination rate, and rate and/or degree of absorption from the route/site of administration.

In one aspect, the invention provides use of a compound of Formula (1) for preparation of a medicament for treating and/or mitigating one or more of viral infection, cardioprotection, stroke, coronary heart disease, myocardial infarction, a neurodegenerative disease, Parkinsonism, Huntington's Chorea, Alzheimer's disease, cardiomyopathy, fatty liver diseases, non-alcoholic fatty liver diseases, and alcohol-related liver disease. In one embodiment cardiomyopathy comprises ischemic, viral, and diabetic cardiomyopathy.

A method is provided of reducing or inhibiting viral infection, stroke, coronary heart disease, myocardial infarction, a neurodegenerative disease, Parkinsonism, Huntington's Chorea, Alzheimer's disease, cardiomyopathy, fatty liver diseases, non-alcoholic fatty liver diseases, and alcohol-related liver disease, comprising administering to a subject in need thereof a pharmaceutical composition comprising an effective amount of a compound of Formula (1). In one aspect, the invention provides a method of providing cardioprotection.

The term “effective amount” encompasses the term “dose” or “dosage,” and is intended to refer to the quantity of pharmaceutically active ingredient administered to the individual in need thereof capable of producing the desired therapeutic effect. The term may refer to a single one-time dose, in a physically discrete unit, such as, for example, in a pill or injection or may refer to multiple doses in physically discrete units. The term “effective amount” also encompasses the quantity of pharmaceutically active ingredient administered to the individual, expressed as the number of molecules, moles, grams, or volume per unit body mass of the individual, such as, for example, mol/kg, mg/kg, ng/kg, ml/kg, or the like, sometimes referred to as concentration administered. The effective amount of pharmaceutically active ingredient may vary among individuals and may fluctuate within an individual over time, depending on factors such as, but not limited to, the condition being treated, genetic profile, metabolic rate, biotransformation capacity, frequency of administration, formulation administered, elimination rate, and rate and/or degree of absorption from the route/site of administration.

Compounds of the invention can be formulated to ensure proper distribution in vivo. For example, therapeutic compounds of the invention can be formulated in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., Ranade, V. V. J. Clin. Pharmacol. (1989) 29 (8): 685-94). Exemplary targeting moieties include folate and biotin (see, e.g., U.S. Pat. No. 5,416,016 to Low et al.); mannosides (Umezawa et al., Biochem. Biophys. Res. Commun. (1988) 153 (3): 1038-44; antibodies (Bloeman et al., FEBS Lett. (1995) 357:140; Owais et al., Antimicrob. Agents Chemother. (1995) 39 (1): 180-4); and surfactant protein A receptor (Briscoe et al., Am. J. Physiol. (1995) 268 (3 Pt 1): L374-80). Liposomal formulations of apoptosis inhibitors may include a targeting moiety.

To administer a therapeutic compound by other than parenteral administration, it may be necessary to coat the compound with, or co-administer the compound with, a material to prevent its inactivation. For example, the therapeutic compound may be administered to a subject in an appropriate vehicle, for example, liposomes, or a diluent. Pharmaceutically acceptable diluents include saline and aqueous buffer solutions. Liposomes include water-in-oil-in-water CGF emulsions as well as conventional liposomes (Strejan et al., Prog. Clin. Biol. Res. (1984) 146:429-34).

The therapeutic compound may also be administered ocularly, via inhalation, topically, intravaginally, as well as parenterally (e.g., intramuscularly, intravenously, intraperitoneally, intraspinally, intrathecally, or intracerebrally). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g. vegetable oil). The proper fluidity can be maintained, for example, by the use of a coating such as lecithin, by the maintenance of the required particle size in the case of dispersion, and by the use of surfactants.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compound is mixed with at least one inert, pharmaceutically acceptable excipient or diluent or assimilable edible vehicle such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compound in the compositions and preparations may, of course, be varied. The amount of the therapeutic compound in such therapeutically useful compositions is such that a suitable dosage will be obtained.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Therapeutic compounds can be administered in time-release or depot form, to obtain sustained release of the therapeutic compounds over time. The therapeutic compounds of the invention can also be administered transdermally (e.g., by providing the therapeutic compound, with a suitable vehicle, in patch form).

It is especially advantageous to formulate parenteral compositions in dosage unit form for case of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention is dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compounds according to the invention are administered at a therapeutically effective dosage sufficient to achieve the desired therapeutic effect, e.g. to prevent, treat and/or mitigate cardioprotection, stroke, coronary heart disease, neurodegenerative diseases, Parkinsonism, Huntington's Chorea, Alzheimer's disease, diabetic cardiomyopathy, fatty liver diseases, non-alcoholic fatty liver diseases, or alcohol-related liver disease. Actual dosage levels of active ingredients in the pharmaceutical compositions may be varied so as to obtain an amount of the active compound(s) that is effective to achieve and maintain the desired therapeutic response for a particular subject, composition, and mode of administration. The selected dosage level will depend upon the activity of the particular compound, the route of administration, frequency of administration, the severity of the condition being treated, the condition and prior medical history of the subject being treated, the age, sex, weight and genetic profile of the subject, and the ability of the therapeutic compound to produce the desired therapeutic effect in the subject. Dosage regimens can be adjusted to provide the optimum therapeutic response. For example, several divided doses may be administered daily or the dose may be proportionally reduced as indicated by the exigencies of the therapeutic situation.

However, it is known within the medical art to determine the proper dose for a particular patient by the dose titration method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed. Also, dosages are routinely determined through preclinical ADME toxicology studies and subsequent clinical trials as required by the FDA or equivalent agency. The ability of an AIF inhibitor to produce the desired therapeutic effect may be demonstrated in various models for the various conditions treated with these therapeutic compounds.

The dosage of active ingredient in the compositions of this invention may be varied; however, it is necessary that the amount of the active ingredient be such that a suitable dosage form is obtained. The selected dosage depends upon the desired therapeutic effect, on the route of administration, and on the duration of the treatment. In general, an effective dosage for the activities of this invention will contain between 1 and 1000 mg, and usually between 5 and 500 mg, of at least one compound of the disclosure, e.g., about 10, 25, 50, 100, 200, 300 or 400 mg per unit dosage which can be administered as a single dose or divided into multiple doses.

As evidenced by Mordenti (J. Pharm. Sci. 1986 75 (11): 1028-40) and similar articles, dosage forms for animals such as, for example, rats can be and are widely used directly to establish dosage levels in therapeutic applications in higher mammals, including humans. In particular, the biochemical cascade initiated by many physiological processes and conditions is generally accepted to be identical in mammalian species (see, e.g., Mattson et al. Neurotrauma 1994 11 (1): 3-33; Higashi et al. Neuropathol. Appl. Neurobiol. 1995 21:480-483). Accordingly, pharmacological agents that are efficacious in animal models such as those described herein are believed to be predictive of clinical efficacy in humans, after appropriate adjustment of dosage.

According to the FDA, calculating a human equivalent dose from animal studies needs to done by normalizing to bovine serum albumin (Center for Drug Evaluation and Research, Center for Biologics Evaluation and Research. (2002) Estimating the safe starting dose in clinical trials for therapeutics in adult healthy volunteers, U.S. Food and Drug Administration, Rockville, Maryland, USA). This can be done using Kₘ factors where the Human equivalent dose (HED)=animal dose in mg/kg multiplied by animal Km/human Kₘ (Reagan-Shaw et al. FASEB J. 2008 March; 22 (3): 659-61). The HED for a dose of 0.05 mg/kg in a mouse is equal to 0.05 mg/kg×3/37=0.004 mg/kg in a human.

One compound, inhibited cell apoptosis induced by HCoV-OC43 (a human coronavirus) in BEAS-2B cells, which is a normal human bronchial epithelium cell line (see Example 1). A BEAS-2B cell line was used for these studies.

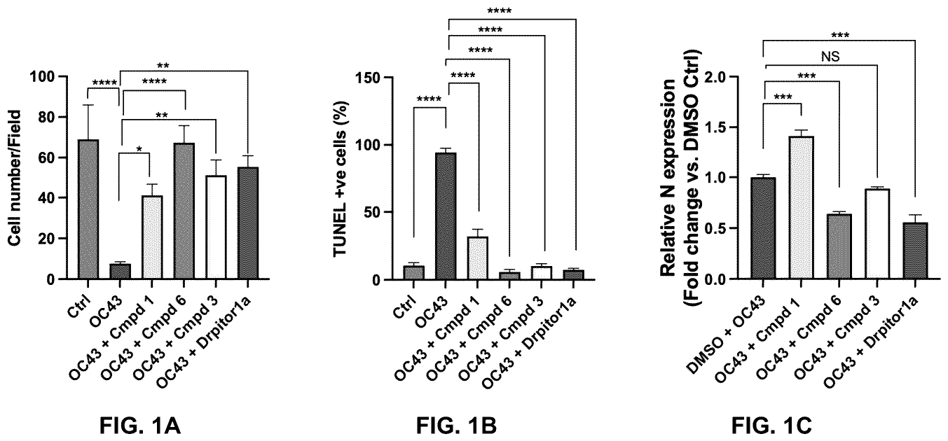

Referring to FIG. 1A, a bar graph shows results of an in vitro study of BEAS-2B cells that were infected with OC43 virus. As indicated by the OC43-only data, cells were killed by infection with this human coronavirus. Then, as shown by the AIF inhibitor bars, cell numbers were preserved by the treatment, indicating protection from cell death. Notably, the AIF inhibitors increased cell number (i.e., prevented cell death) upon challenge with the human coronavirus HCoVOC43.

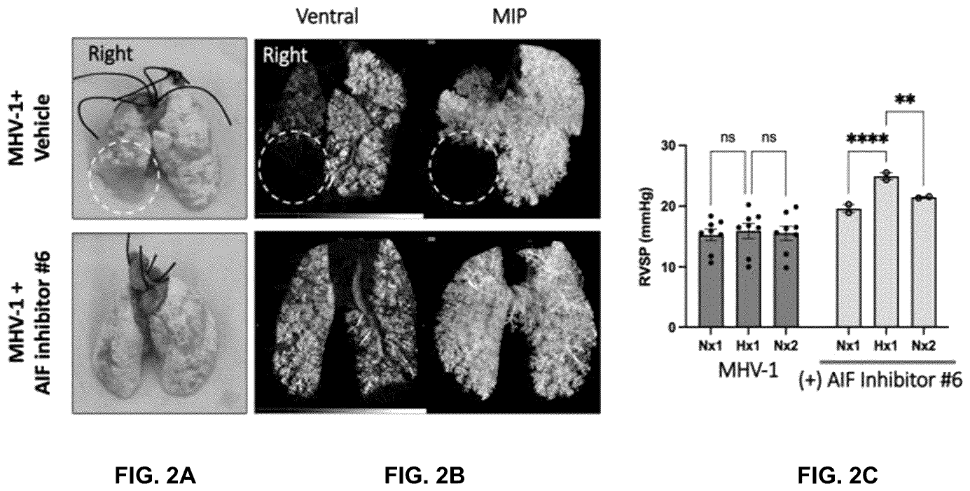

As shown in FIGS. 2A-C, AIF Inhibitor 6 reduced lung edema (see FIG. 2A), increased lung perfusion (see FIG. 2B) and restored hypoxic pulmonary vasoconstriction as shown by a rise in RVSP with hypoxia (see FIG. 2C). Referring to FIGS. 2A, the treated murine lungs had significantly less structural damage. This lack of damage (i.e., lung edema) was also apparent in the images of FIG. 2B, which shows more homogenous perfusion of the lung by the trachea. Referring to FIG. 2B, micro-CT images are shown of the same lungs as in FIG. 2A. In the column labelled Ventral, the image was taken from the belly side of the mouse. In the column labelled Maximum Intensity Projection (MIP), images were collected using this mode to better shows the barium contrast agent, which indicates open airways. Notably, the yellow circle indicates an area of lack of perfusion in the right lower lobe of the control lungs that was caused by damage from the infection. The bottom lungs are from the mouse treated with compound 6 and does not have a damaged area. This result is also shown graphically in FIG. 2C, with a bar graph that shows hemodynamics of RVSP, where the left three bars are from one mouse that was treated with MHV-1 and vehicle only (control, Ctrl). The mouse was provided with room air for ten minutes (Nx1, where Nx refers to normoxia), then hypoxic air (10% O₂) for ten minutes (Hx1, where Hx refers to hypoxia), and then the animal was provided room air again for ten minutes (Nx2). The right three bars are from one mouse that was treated with MHV-1 plus AIF Inhibitor 6. It was provided with room air for 10 minutes (Nx1), then hypoxic air (10% O₂) for ten minutes (Hx1), and then the animal was provided room air again for ten minutes (Nx2). The results show the control animal was unable to show normal behavior in hypoxic conditions, namely vasoconstriction of the small pulmonary arteries resulting in a spike in RVSP. The treated mouse was able to show normal behavior in hypoxic conditions. Preservation of hypoxic pulmonary vasoconstriction, a mitochondrial mediated oxygen sensor response, increased systemic oxygenation (see Archer, S. J. et al., Redox Biology 58 (2022) 102508). That is, the AIF inhibitor restored the hypoxic rise in right ventricular systolic pressure. This rise shows restoration of hypoxic pulmonary vasoconstriction, the lungs autoregulatory response to pneumonia and hypoxia that optimizes oxygen delivery.

Studies described herein show efficacy of AIF and mitochondrial fission inhibitors in reducing apoptosis without increasing viral load. It was determined that HCoV-OC43 infection reduced cell numbers, which was reversed by AIF inhibitors and Drpitor1a (see FIG. 1A). Apoptosis was reduced by three novel AIF inhibitors (1, 3, and 6) and fission inhibitor Drpitor1a. Drpitor1a is an inhibitor of dynamin related protein 1, Drp1, which has been shown to prevent mitochondrial fission (see U.S. Pat. No. 11,229,629). Accordingly, these compounds reduced viral load.

The following working examples further illustrate the invention and are not intended to be limiting in any respect.

### Working Examples

### Methods

Cell Culture: BEAS-2B cells (an epithelial cell line that was isolated from normal human bronchial epithelium) were purchased from the American Type Culture Collection (ATCC) through Cedarlane Corp., Burlington, Canada. The cells were cultured in bronchial epithelial cell growth medium (Lonza/Clonetics Corporation, Basel, Switzerland) supplemented with 10% fetal bovine serum (FBS). Cells were cultured at 37° C. in a humidified incubator balanced with 5% CO₂.

Compound solution preparation for in vitro experiments: Candidate compounds were dissolved in anhydrous Dimethyl Sulfoxide (DMSO) (available from Sigma Aldrich), filtered by a 0.20 μm PTFE filter and added to culture medium to make a working solution at designated concentrations. Final concentration of DMSO in culture medium was ≤0.1%. Same volume of DMSO was used as control.

Statistical analyses: Quantitative data are presented as mean±standard error of mean (SEM). Student's t-test or Mann-Whitney U test was used to compare the mean or median value between two groups as appropriate. One-way ANOVA was used to compare the means of three or more independent groups. Two-way ANOVA was used to compare the mean differences between groups that have two independent variables. A P-value of less than 0.05 was considered statistically significant.

### Example 1. Apoptosis Studies

An apoptosis study was conducted (see FIGS. 1A-1C). HCoV-OC43 infection (referred to as “OC43” herein) see Archer, S. L., et al., Redox Biology (2002) 58:102508) was performed in BEAS-2B cells (an epithelial cell line that was isolated from normal human bronchial epithelium, available from ATCC through Cedarlane Corp., Burlington, Canada). Cells were counted and then seeded in a cell culture plate overnight. The cell culture media was replaced with serum-free media containing 3 focus-forming units (ffu)/cell of OC43 virus. The cells were cultured in this inoculum for 2 hours. Then the inoculum was replaced with complete growth medium and incubated at 33° C. until 72 hours post-infection (the time of the experimental endpoint). Cells were then treated for 48 hours with AIF Inhibitor compounds starting at 24 hours post infection. Concentration of compounds: AIF inhibitor 1, AIF inhibitor 3, and AIF inhibitor 6 were used at a concentration of 10 uM; Drpitor1a, which is a Drp1 inhibitor and is not an AIF inhibitor) was used at a concentration of 1 uM. Cell viability was measured by cell count (see FIG. 1A). Apoptotic cells were measured by TUNEL staining (see FIG. 1B) (see Archer, S. L., et al., Redox Biol. (2022) 58:102508). Cells were dual stained with Click-iT® Plus TUNEL (available from Thermo Fisher, Waltham, MA, USA) and 4′,6-diamidino-2-phenylindole (DAPI) (available from Thermo Fisher, Waltham, MA, USA). Images were captured on a Leica SP8 confocal microscope using a 63×1.4 NA objective and a white light laser at 488 nm. Emission acquisition gating was 510-530 nm (for TUNEL). A 405 nm laser diode was used for DAPI excitation and the emission acquisition gate was set at 420-480 nm. Viral loads were quantified using qRT-PCR (see FIG. 1C). mRNA level of HCoV-OC43 N protein was measured and compared among treatment groups.

Efficacy of AIF inhibitors and a mitochondrial fission inhibitor (Drpitor1a) in reducing apoptosis without increasing viral load were evaluated. It was determined that HCoV-OC43 infection reduced cell numbers, which was reversed by AIF inhibitors and Drpitor1a (see FIG. 1A). Apoptosis was reduced by three novel AIF inhibitors (compounds 1, 3, and 6) and fission inhibitor Drpitor1a. Accordingly, these compounds reduced viral load.

### Example 2. Murine Model of MHV-1 Pneumonia (Model and Hemodynamics)

Five-week-old A/J mice (Jackson Labs, Bar Harbour, Maine) were given an intranasal inoculation of either MHV-1 (5000 PFU/17 μl) or an identical volume of saline as control. AIF inhibitor 6 was administered for 5 days (1 mg/kg, QD, IP) post infection. At day 5 post-infection, mice were anesthetized with a single IP injection of ketamine/xylazine (150 mg/kg and 10 mg/kg, respectively). O₂ saturation was measured while mice breathed room air, following which they were intubated and mechanically ventilated, which increased their O₂ saturation. Transcutaneous O₂ saturation was monitored during the procedure using a MouseSTAT® Jr. rodent pulse oximeter attached to the rear paw (Kent Scientific Corporation, Torrington, CT, USA). Right heart catheterization (RHC) was performed using a 1.2F Scisense pressure-volume catheter (Transonic Systems Inc., Ithaca, NY, USA) via a closed chest approach. Mice were ventilated with normoxic gas (room air, 20.6% O₂) for 10 minutes, followed by hypoxic gas for 10 minutes (10% 02 balanced with N₂) and then followed by normoxic gas (room air, 20.6% O₂) for 10 minutes. This regimen was designed to elicit hypoxic pulmonary vasoconstriction (HPV), a normal physiologic response of the pulmonary arteries to airway hypoxia.

### Micro-Computed Tomography (Micro-CT):

Referring to FIGS. 2A-2B, photographs and micro-CT images are shown from a study probing the effect of AIF Inhibitors on murine lung that had been exposed to MHV-1. Lungs were fixed with 4% formaldehyde (PFA) and perfused with 25% barium sulfate via the trachea and imaged by microCT. The micro-CT images were acquired using a VECTor4CT pre-clinical scanner (MILabs B.V., Utrecht, Netherlands) equipped with a cone beam X-ray CT system. The measurements used an acceleration voltage of 50 kVp and X-ray tube current of 430 μA in the ultra-focus mode, allowing acquisition of 3,600 projections with exposure time 40 ms, resulting in a 25 μm resolution. Tomographic CT images of FIG. 2B were compiled using MILabs reconstruction software. For quantification and segmentation, CT image data was manually processed using PMOD 3.9 software (PMOD Technologies Ltd., Zurich, Switzerland).

### EQUIVALENTS

It will be understood by those skilled in the art that this description is made with reference to certain embodiments and that it is possible to make other embodiments employing the principles of the invention which fall within its spirit and scope.

TABLE 1
Names and Structural Formulae

| Name | Structure |
|---|---|
| 2-[(1-methyl-1,4-dihydroquinazolin-2-yl)methyl)-6- (tetrahydro-2H-pyran-4-yl)-5,6-dihydropyrimidin- 4(3H)-one (1) | 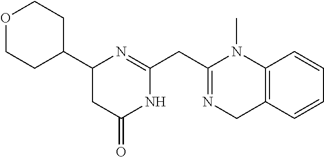 [SMILES: CN1C(CC2=NC(C3CCOCC3)CC(=O)N2)=NCc2ccccc21] |
| | (1) |
| 2-benzhydryl-4,5,7-trifluorobenzo[d]thiazole (2) | 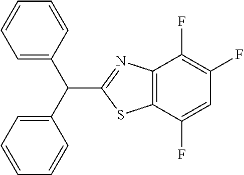 [SMILES: Fc1cc(F)c2sc(C(c3ccccc3)c3ccccc3)nc2c1F] |
| | (2) |
| N,N-diethyl-3-(2-oxo-1,2-dihydroquinoline-4- carbonyl)quinoline-2-carboxamide (3) | 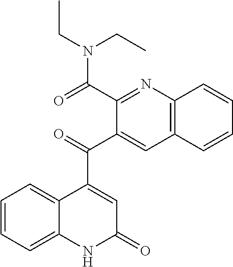 [SMILES: CCN(CC)C(=O)c1nc2ccccc2cc1C(=O)c1cc(=O)[nH]c2ccccc12] |
| | (3) |
| 5-cyano-11H-benzo[a]carbazol-6-yl trifluoromethanesulfonate (4) | 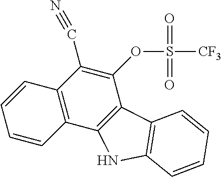 [SMILES: N#Cc1c(OS(=O)(=O)C(F)(F)F)c2c3ccccc3[nH]c2c2ccccc12] |
| | (4) |
| 1-(3-bromopropyl)-4-(3- (cyclohexyloxy)propoxy)benzene (5) | 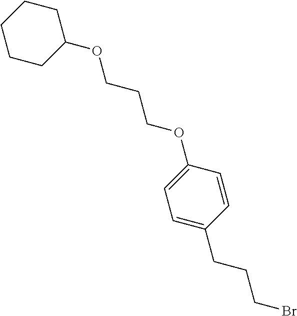 [SMILES: BrCCCc1ccc(OCCCOC2CCCCC2)cc1] |
| | (5) |
| 2-(2-(5-(benzyloxy)-1H-indol-3-yl)ethyl) isoindoline-1,3-dione (6) | 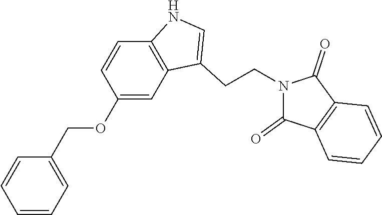 [SMILES: O=C1c2ccccc2C(=O)N1CCc1c[nH]c2ccc(OCc3ccccc3)cc12] |
| | (6) |
| Drpitor1a | 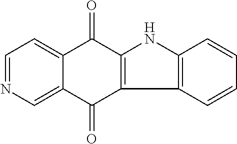 [SMILES: O=C1c2ccncc2C(=O)c2c1[nH]c1ccccc21] |

TABLE 2
Identification of candidate compounds from in
silico screening and predicted binding affinity
and structural formula of compounds of FIG. 1

| Compound | 1 | 2 | 3 | 4 | 5 | 6 |
|---|---|---|---|---|---|---|
| Lipinsky Rule of 5 | ✓ | ✓ | ✓ | ✓ | ✓ | ✓ |
| Aromatic Ring Count | 2 | 4 | 4 | 4 | 1 | 4 |
| Predicted Solubility | −3.0 | −6.3 | −5.2 | −6.7 | −5.0 | −6.3 |
| (Log S)* | | | | | | |
| Lipophilicity (Log | 1.8 | 6.8 | 2.6 | 4.3 | 4.9 | 4.1 |
| P)^{†} | | | | | | |

*Log S values are in the range −10 to +2; values >−4 indicate optimal solubility
^{†}Log P values: for oral and intestinal absorption the ideal value is 1.35-1.8.

1. A compound selected from:
2-[(1-methyl-1,4-dihydroquinazolin-2-yl)methyl)-6-(tetrahydro-2H-pyran-4-yl)-5,6-dihydropyrimidin-4 (3H)-one (1);
2-benzhydryl-4,5,7-trifluorobenzo[d]thiazole (2);
N,N-diethyl-3-(2-oxo-1,2-dihydroquinoline-4-carbonyl) quinoline-2-carboxamide (3);
5-cyano-11H-benzo[a]carbazol-6-yl trifluoromethanesulfonate (4); or
1-(3-bromopropyl)-4-(3-(cyclohexyloxy)propoxy)benzene (5)

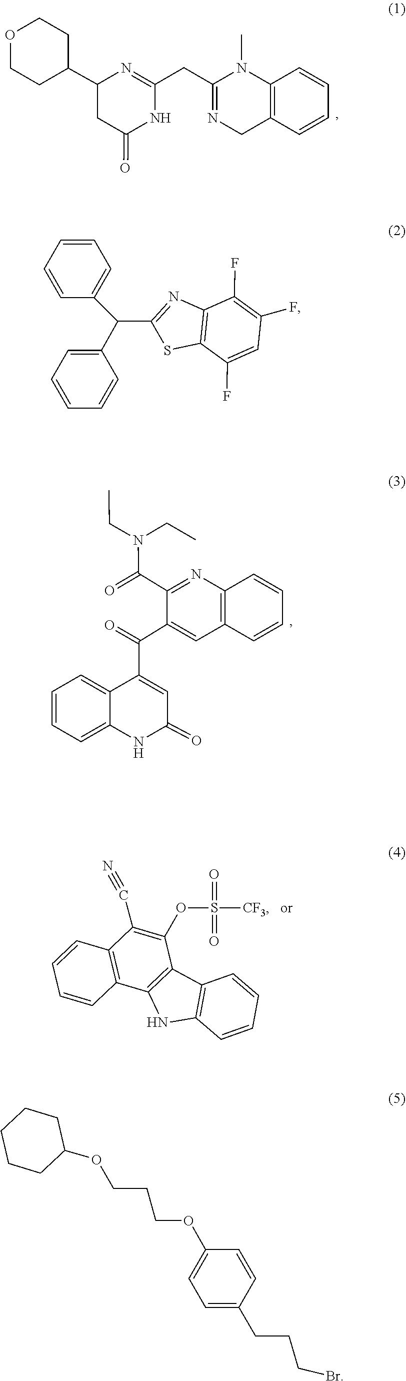

2. A pharmaceutical composition comprising a compound selected from the group consisting of:
2-[(1-methyl-1,4-dihydroquinazolin-2-yl)methyl)-6-(tetrahydro-2H-pyran-4-yl)-5,6-dihydropyrimidin-4 (3H)-one (1);
2-benzhydryl-4,5,7-trifluorobenzo[d]thiazole (2);
N,N-diethyl-3-(2-oxo-1,2-dihydroquinoline-4-carbonyl) quinoline-2-carboxamide (3);
5-cyano-11H-benzo[a]carbazol-6-yl trifluoromethanesulfonate (4);
1-(3-bromopropyl)-4-(3-(cyclohexyloxy)propoxy)benzene (5); and
2-(2-(5-(benzyloxy)-1H-indol-3-yl)ethyl) isoindoline-1,3-dione (6); 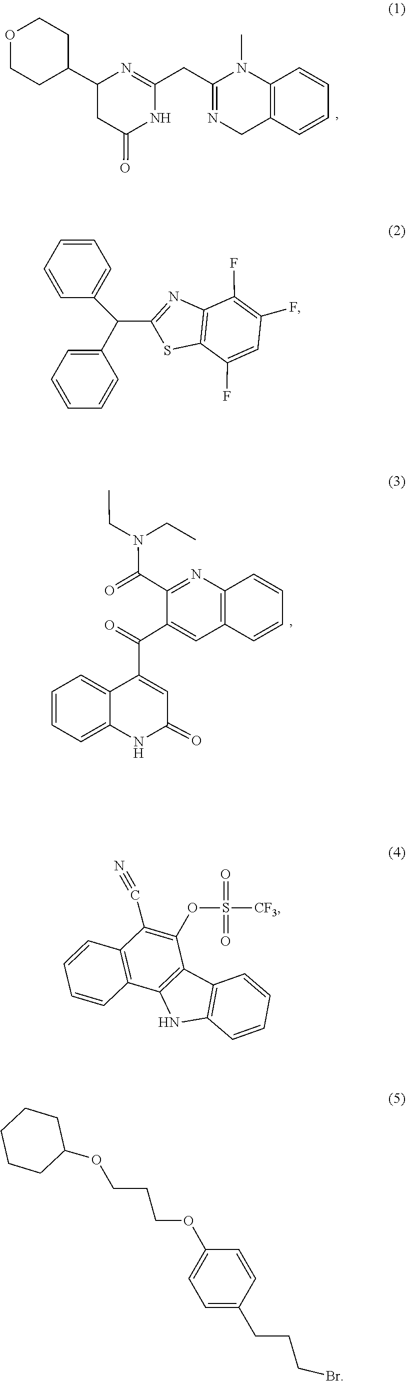 [SMILES: CN1C(CC2=NC(C3CCOCC3)CC(=O)N2)=NCc2ccccc21 ; Fc1cc(F)c2sc(C(c3ccccc3)c3ccccc3)nc2c1F ; CCN(CC)C(=O)c1nc2ccccc2cc1C(=O)c1cc(=O)[nH]c2ccccc12 ; N#Cc1c(OS(=O)(=O)C(F)(F)F)c2c3ccccc3[nH]c2c2ccccc12 ; BrCCCc1ccc(OCCCOC2CCCCC2)cc1] 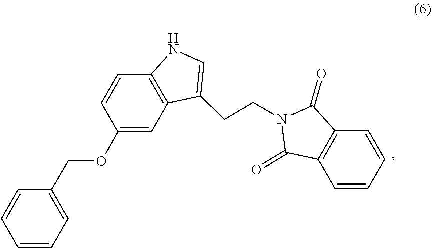 [SMILES: O=C1c2ccccc2C(=O)N1CCc1c[nH]c2ccc(OCc3ccccc3)cc12]
or a pharmaceutically acceptable salt thereof,
for inhibiting apoptosis in a subject.

3. The pharmaceutical composition of claim 2, wherein the pharmaceutical composition inhibits apoptosis by inhibiting activity of apoptosis inducing factor (AIF) in the subject.

4. The pharmaceutical composition of claim 2, wherein the compound is compound (1), (3), or (6), or a pharmaceutically acceptable salt thereof.

5. The pharmaceutical composition of claim 2, for the treatment and/or mitigation of one or more of a viral infection including infections mediated by coronaviruses, including SARS-CoV-2 and HCOV-OC43, and Herpes viruses, stroke, coronary heart disease, myocardial infarction, ischemia-reperfusion injuries, as occurs in cardiac arrest, neurodegenerative disease, Parkinsonism, Huntington's Chorea, Alzheimer's disease, cardiomyopathies (including ischemic, viral, and diabetic cardiomyopathy), fatty liver diseases, non-alcoholic fatty liver diseases, and alcohol-related liver disease.

6. The pharmaceutical composition of claim 2, for providing cardioprotection.

7. The pharmaceutical composition of claim 2, comprising a pharmaceutically acceptable vehicle.

8. The pharmaceutical composition of claim 6, wherein the pharmaceutically acceptable vehicle is an excipient.

9. The pharmaceutical composition of claim 2, wherein the compound is present in an amount from 1 mg to 1000 mg.

10. The pharmaceutical composition of claim 2, wherein the compound is present in an amount from 5 mg to 500 mg.

11. The pharmaceutical composition of claim 2, wherein the subject is human.

12. A method for inhibiting apoptosis in a subject comprising administering to the subject an effective amount of the pharmaceutical composition of claim 2.

13. The method of claim 12, wherein the compound is compound (1), (3), or (6), or a pharmaceutically acceptable salt thereof.

14. The method of claim 12, wherein the subject is human.

15. A method for inhibiting apoptosis inducing factor (AIF) in a subject comprising administering to the subject an effective amount of the pharmaceutical composition of claim 2.

16. The method of claim 15, wherein the compound is compound (1), (3), or (6), or a pharmaceutically acceptable salt thereof.

17. The method of claim 15, wherein the subject is human.

18. Use of a compound of claim 1 in the preparation of a medicament for treating and/or mitigating one or more of a viral infection, stroke, coronary heart disease, myocardial infarction, neurodegenerative disease, Parkinsonism, Huntington's Chorea, Alzheimer's disease, cardiomyopathy, fatty liver disease, non-alcoholic fatty liver disease, and alcohol-related liver disease, or for providing cardioprotection.

19. The use of claim 18, wherein the viral infection is a virus selected from the group consisting of coronavirus, SARS-CoV-2, HCOV-OC43, and Herpes virus.

20. The use of claim 18, wherein the cardiomyopathy is ischemic, viral, or diabetic cardiomyopathy.
